(19) United States
(12) Patent Application Publication
(10) Pub. No.: US 2020/0388058 A1
(43) Pub. Date: Dec. 10, 2020

(54) DEEP LEARNING BASED IMAGE FIGURE OF MERIT PREDICTION

(71) Applicant: KONINKLIJKE PHILIPS N.V., EINDHOVEN (NL)

(72) Inventors: Bin ZHANG, CLEVELAND, OH (US); Binit PANDA, HIGHLAND HEIGHTS, OH (US); Chuanyong BAI, SOLON, OH (US); Zhiqiang HU, TWINSBURG, OH (US)

(21) Appl. No.: 16/961,948

(22) PCT Filed: Jan. 15, 2019
(86) PCT No.: PCT/EP2019/050869

§ 371 Date: Jul. 14, 2020

Related U.S. Application Data
(60) Provisional application No. 62/620,091, filed on Jan. 22, 2018.

(51) Int. Cl. G06T 11/00; A61B 6/03; G06N 3/08

(52) U.S. Cl. CPC G06T 11/005; G06T 2210/41; G06N 3/08; A61B 6/037

(57) ABSTRACT

A non-transitory computer-readable medium stores instructions readable and executable by a workstation (18) including at least one electronic processor (20) to perform an imaging method (100). The method includes: estimating one or more figures of merit for a reconstructed image by applying a trained deep learning transform (30) to input data including at least imaging parameters and not including a reconstructed image; selecting values for the imaging parameters based on the estimated one or more figures of merit; generating a reconstructed image using the selected values for the imaging parameters; and displaying the reconstructed image.

### FIELD

The following relates generally to the medical imaging arts, medical image interpretation arts, image reconstruction arts, and related arts.

### BACKGROUND

Positron Emission Tomography (PET) imaging provides critical information for oncology and cardiology diagnosis and treatment planning Two classes of figures of merits are important to clinical usage of PET images: qualitative figures of merits such as noise level of the image, and quantitative ones such as the lesion Standardized Uptake Value (SUV) and contrast recovery ratio. In PET imaging, these figures of merits are measured on the images reconstructed from acquired data sets. The obtained figures of merits of the images are end result of an imaging chain, which provides no or limited feedback to the image chain that generates the image.

A user generally cannot predict how much a given figure of merit will change if some parameters change (e.g. patient weight, scan time, or reconstruction parameters). A common way to solve this issue is to use a try-and-see approach by performing many reconstructions for each individual case. With many attempts, a user gains an idea about correlations. However, the reconstruction can take on the order of 5-10 minutes for a high resolution image, so that this process can take quite some time and effort.

When imaging parameters comprising image acquisition parameters are to be adjusted the difficulty is even greater. In the case of an imaging modality such as ultrasound, the process of acquiring imaging data and reconstructing an image is rapid, so that adjusting an ultrasound imaging data acquisition parameter based on the reconstructed ultrasound image is a practical approach. However for PET, such a try-and-see approach for adjusting acquisition parameters can be impractical. This is because PET imaging data acquisition must be timed to coincide with residency of an administered radiopharmaceutical in tissue of the patient which is to be imaged. Depending upon the half-life of the radiopharmaceutical and/or the rate at which the radiopharmaceutical is removed by action of the kidneys or other bodily functions, the PET imaging data acquisition time window can be narrow. Furthermore, the dosage of radiopharmaceutical is usually required to be kept low to avoid excessive radiation exposure to the patient, which in turn requires relatively long imaging data acquisition times in order to acquire sufficient counts for reconstructing a PET image of clinical quality. These factors can preclude the try-and-see approach of acquiring PET imaging data, reconstructing the PET image, adjusting PET imaging data acquisition parameters based on the reconstructed PET image, and repeating.

The following discloses new and improved systems and methods to overcome these problems.

### SUMMARY

In one disclosed aspect, a non-transitory computer-readable medium stores instructions readable and executable by a workstation including at least one electronic processor to perform an imaging method. The method includes: estimating one or more figures of merit for a reconstructed image by applying a trained deep learning transform to input data including at least imaging parameters and not including a reconstructed image; selecting values for the imaging parameters based on the estimated one or more figures of merit; generating a reconstructed image using the selected values for the imaging parameters; and displaying the reconstructed image.

In another disclosed aspect, an imaging system includes a positron emission tomography (PET) image acquisition device configured to acquire PET imaging data. At least one electronic processor is programmed to: estimate one or more figures of merit for a reconstructed image by applying a trained deep learning transform to input data including at least image reconstruction parameters and statistics of imaging data and not including the reconstructed image; select values for the image reconstruction parameters based on the estimated one or more figures of merit; generate the reconstructed image by reconstructing the imaging data using the selected values for the image reconstruction parameters; and control a display device to display the reconstructed image.

In another disclosed aspect, an imaging system includes a positron emission tomography (PET) image acquisition device configured to acquire PET imaging data. At least one electronic processor is programmed to: estimate one or more figures of merit for a reconstructed image by applying a trained deep learning transform to input data including at least image acquisition parameters and not including the reconstructed image; select values for the image acquisition parameters based on the estimated one or more figures of merit; generate the reconstructed image by acquiring imaging data using the image acquisition device with the selected values for the image acquisition parameters and reconstructing the acquired imaging data to generate the reconstructed image; and control a display device to display the reconstructed image.

One advantage resides in providing an imaging system the generates a priori predictions on outcomes of targeted figures of merit (e.g., general image noise levels, standard uptake value (SUV) recovery) before expending computational resources in performing complex image reconstruction.

Another advantage resides in using targeted figures of merit to design an imaging protocol.

Another advantage resides in assessing figures of merit that can be achieved by different reconstruction methods and parameters but without the need to perform complex image reconstruction of the data set.

Another advantage resides in making fast predictions on imaging outcomes when specification of a patient change (e.g., weight loss).

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows imaging system according to one aspect;

FIG. 2 shows an exemplary flow chart operation of the system of FIG. 1;

FIG. 3 shows an exemplary flow chart training operation of the system of FIG. 1; and

FIGS. 4 and 5 show exemplary flow chart operations of the system of FIG. 1.

### DETAILED DESCRIPTION

Current high resolution image reconstruction takes 5-10 minutes for an image dataset, and acquisition takes much longer than this. Typically, an imaging session will employ default parameters for the imaging acquisition (e.g. default radiopharmaceutical dose per unit weight, default wait time between administration of the radiopharmaceutical and commencement of PET imaging data acquisition, default acquisition time per frame, et cetera) and default reconstruction parameters. It is desired that image figures of merit such as noise level in the liver, a mean standard uptake value (SUV mean) in tumors, contrast recovery ratio of a lesion, or so forth will fall within certain target ranges. If this is not the case, then either the clinical interpretation is performed with substandard reconstructed images or the image reconstruction (or even acquisition) must be repeated with improved parameters. Moreover, it may be difficult to determine which direction to adjust a given parameter to improve the image figure(s) of merit. In the case of adjusting parameters of the image reconstruction, each adjustment is followed by a repetition of the image reconstruction, which as noted may take 5-10 minutes per iteration. In the case of imaging data acquisition parameters, it is generally not advisable to repeat the PET imaging data acquisition as such a repetition would require administering a second radiopharmaceutical dose.

The following disclosed embodiments leverage deep learning of a Support Vector Machine (SVM) or neural network (NN) that is trained to predict the figure(s) of merit based on standard inputs, which do not include the reconstructed image.

In some embodiments disclosed herein, the inputs to the SVM or neural network include solely information available prior to imaging data acquisition, such as patient weight and/or body mass index (BMI) and the intended (default) imaging parameters (e.g. acquisition parameters such as dose and wait time, and image reconstruction parameters). The SVM or neural network is trained on training instances each comprising the input (training) PET imaging data paired with actual figure(s) of merit derived from the corresponding reconstructed training images. The training optimizes the SVM or neural network to output the figure(s) of merit optimally matching the corresponding figure of merit values measured for the actual reconstructed training images. In application, the available input for a scheduled clinical PET imaging session is fed to the trained SVM or neural network which outputs predictions of the figure(s) of merit. In a manual approach the predicted figure(s) of merit are displayed, and if the predicted values are unacceptable to the clinician he or she can adjust the default imaging parameters and re-run through the SVM or neural network in an iterative fashion until desired figure(s) of merit are achieved. Thereafter, the PET imaging is performed using the adjusted imaging parameters, with a high expectation that the resulting reconstructed image will likely exhibit the desired figure(s) of merit.

In other embodiments disclosed herein, the figure of merit prediction is performed after the imaging data acquisition but prior to image reconstruction. In these embodiments, the inputs to the SVM or neural network further include statistics of the already-acquired imaging data set, e.g. the total counts, counts/minute or so forth. The training likewise employs these additional statistics for the training imaging data sets. The resulting trained SVM or neural network can again be applied after the imaging data acquisition but prior to commencement of image reconstruction, and is likely to provide more accurate estimation of the figure(s) of merit due to the additionally provided statistical information. In this case since the imaging data are already acquired the imaging parameters to be optimized are limited to the image reconstruction parameters.

The disclosed embodiments improve imaging and computational efficiency by enabling imaging parameters (e.g. acquisition and/or reconstruction parameters) to be optimized before performing any actual image reconstruction, and even before image acquisition in some embodiments.

Although described herein for PET imaging systems, the disclosed approaches can be disclosed in computed tomography (CT) imaging systems, hybrid PET/CT imaging systems; single photon emission computed tomography (SPECT) imaging systems, hybrid SPECT/CT imaging systems, magnetic resonance (MR) imaging systems; hybrid PET/MR, functional CT imaging systems, functional MR imaging systems, and the like.

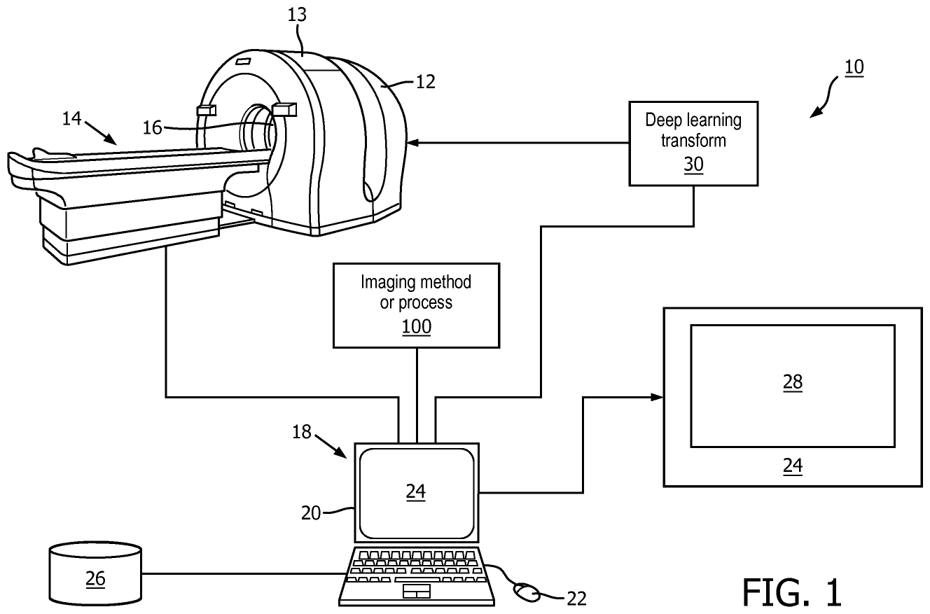

With reference to FIG. 1, an illustrative medical imaging system 10 is shown. As shown in FIG. 1, the system 10 includes an image acquisition device or imaging device 12. In one example, the image acquisition device 12 can comprise a PET imaging device. The illustrative example is a PET/CT imaging device, which also includes a CT gantry 13 suitably used to determine anatomical information and to generate an attenuation map from the CT images for use in correcting for absorption in the PET reconstruction. In other examples, the image acquisition device 12 can be any other suitable image acquisition device (e.g., MR, CT, SPECT, hybrid devices, and the like). A patient table 14 is arranged to load a patient into an examination region 16 of the PET gantry 12.

The system 10 also includes a computer or workstation or other electronic data processing device 18 with typical components, such as at least one electronic processor 20, at least one user input device (e.g., a mouse, a keyboard, a trackball, and/or the like) 22, and a display device 24. In some embodiments, the display device 24 can be a separate component from the computer 18. The workstation 18 can also include one or more non-transitory storage media 26 (such as a magnetic disk, RAID, or other magnetic storage medium; a solid state drive, flash drive, electronically erasable read-only memory (EEROM) or other electronic memory; an optical disk or other optical storage; various combinations thereof; or so forth). The display device 24 is configured to display a graphical user interface (GUI) 28 including one or more fields to receive a user input from the user input device 22.

The at least one electronic processor 20 is operatively connected with the one or more non-transitory storage media 26 which stores instructions which are readable and executable by the at least one electronic processor 20 to perform disclosed operations including performing an imaging method or process 100. In some examples, the imaging method or process 100 may be performed at least in part by cloud processing. The non-transitory storage media 26 further store information for training an implementing a trained deep learning transform 30 (e.g., an SVM or a NN).

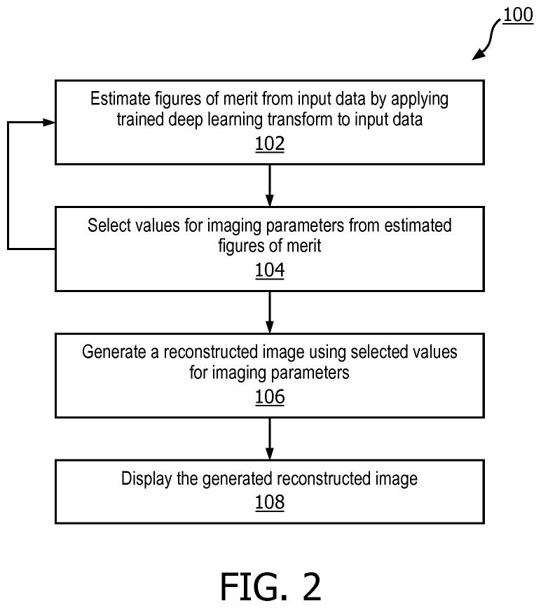

With reference to FIG. 2, an illustrative embodiment of the image reconstruction method 100 is diagrammatically shown as a flowchart. At 102, the at least one electronic processor 20 is programmed to estimate one or more figures of merit for a reconstructed image by applying a trained deep learning transform 30 to input data including at least imaging parameters and not including a reconstructed image. In some embodiments, the trained deep learning transform is a trained SVM or a trained neural network. In one example, the one or more figures of merit include a standardized uptake value (SUV) for an anatomical region. In another example, the one or more figures of merit include a noise level for an anatomical region. Since the trained deep learning transform 30 does not utilize a reconstructed image as input, the figure of merit prediction 102 advantageously can be performed prior to performing computationally intensive image reconstruction.

The input data can include patient parameters (such as weight, height, gender, etc.); imaging data acquisition parameters (e.g., scan duration, uptake time, activity, and so forth); and, in some embodiments, reconstruction parameters (e.g., iterative reconstruction algorithm, number of iterations to be performed, subset number (e.g., in the case of Ordered Subset Expectation Maximization, OSEM, reconstruction), regularization parameters in the case of regularized image reconstruction, smoothing parameters of an applied smoothing filter or regularization, etc.).

In one embodiment, the input data includes imaging parameters comprising at least image reconstruction parameters; statistics of imaging data (e.g., total counts, counts/minute, or so forth); and information available prior to imaging data acquisition, such as patient weight and/or body mass index (BMI), and the intended (default) imaging parameters (e.g. acquisition parameters such as dose and wait time, type of imaging system, imaging system specification (such as crystal geometry, crystal type, crystal size) and so forth). In addition, the input data does not include the imaging data. In this embodiment, the generating includes generating the reconstructed image by reconstructing the imaging data using the selected values for the image reconstruction parameters.

In another embodiment, the input data includes at least image acquisition parameters, and information available prior to imaging data acquisition, such as patient weight and/or BMI, and does not include the acquired imaging data of the statistics of the acquired imaging data. In this embodiment, the generating includes acquiring imaging data using the imaging device 12 with the selected values for the image acquisition parameters and reconstructing the acquired imaging data to generate the reconstructed image.

Existing approaches for generating corrected reconstructed images typically require a reconstructed image as input data for the correction operations. As previously noted, this can be problematic in certain imaging modalities such as PET. In an imaging modality such as ultrasound the imaging data acquisition and reconstruction is rapid, and there is often no limitations preventing multiple imaging data acquisitions. By contrast, PET image reconstruction is computationally complex and can take on the order of 5-10 min in some cases, and PET imaging data acquisition must be timed with residency of a radiopharmaceutical in the tissue to be imaged, which can severely limit the time window during which imaging data acquisition can occur, and is usually a slow process due to the low counts provided by low radiopharmaceutical dosage dictated by patient safety considerations. Advantageously, the embodiments disclosed herein utilize the trained deep learning transform 30 to make a priori predictions on outcomes of targeted figures of merit (e.g., general image noise levels, standard uptake value (SUV) recovery) before expending computational resources in performing complex image reconstruction, and in some embodiments even before acquiring imaging data. In addition, the figures of merit can be estimate by the trained deep learning transform 30 using different reconstruction methods and parameters but without the need to perform complex image reconstruction of the data set. Stated another way, the trained deep learning transform 30 can estimate the figures of merit without needing a reconstructed image as a necessary input parameter (and in some embodiments even without the acquired imaging data).

At 104, the at least one electronic processor 20 is programmed to select values for the imaging parameters based on the estimated one or more figures of merit. To do so, the at least one electronic processor 20 is programmed to compare the estimated one or more figures of merit with target values for the one or more figures of merit (i.e., target values that are stored in the one or more non-transitory storage media 26). The at least one electronic processor 20 is then programmed to adjust the imaging parameters based on the comparing operation. The at least one electronic processor 20 is then programmed to repeat the estimation of the one or more figures of merit for the reconstructed image by applying the trained deep learning transform 30 to input data including at least the adjusted imaging parameters. In some embodiments, the input data does not include a reconstructed image.

At 106, the at least one electronic processor 20 is programmed to generate a reconstructed image by performing image reconstruction of the acquired imaging data using the selected values for the imaging parameters. If the figure of merit prediction/optimization 102, 104 is performed prior to imaging data acquisition, then the step 106 includes acquiring the PET imaging data and then performing reconstruction. On the other hand, if figure of merit prediction/optimization 102, 104 is performed after imaging data acquisition (with the imaging data statistics being inputs to the SVM or NN 30), then the step 106 includes performing the image reconstruction. The step 106 suitably employs the imaging parameters as adjusted by the figure of merit prediction/optimization 102, 104.

At 108, the at least one electronic processor 20 is programmed to control the display device 24 to display the reconstructed image. Additionally, the step 108 may perform figure of merit assessment on the reconstructed image to determine, for example, the noise figure in the liver, SUV values in lesions, and/or other figures of merit. Due to the figure of merit prediction/optimization 102, 104, there is a substantially improved likelihood that the figure(s) of merit assessed from the reconstructed image will be close to the desired values.

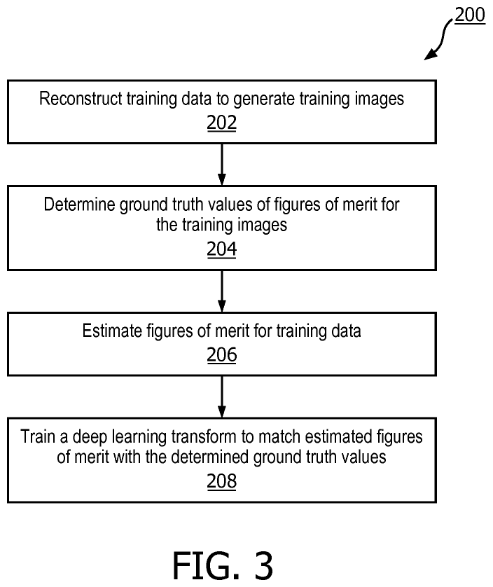

With reference to FIG. 3, an illustrative embodiment of a training method 200 of the trained deep learning transform 30 is diagrammatically shown as a flowchart. At 202, the at least one electronic processor 20 is programmed to reconstruct training imaging data to generate corresponding training images. At 204, the at least one electronic processor 20 is programmed to determine values of the one or more figures of merit for the training images by processing of the training image. At 206, the at least one electronic processor 20 is programmed to estimate the one or more figures of merit for the training imaging data by applying the deep learning transform 30 to input data including at least the image reconstruction parameters and statistics of the training imaging data. At 208, the at least one electronic processor 20 is programmed to train the deep learning transform 30 to match the estimates of the one or more figures of merit for the training imaging data with the determined values. The training 208 may, for example, use backpropagation techniques known for training a deep learning transform comprising a neural network. In the case of training a deep learning transform comprising a Support Vector Machine (SVM), known approaches for optimizing the hyperplane parameters of the SVM are employed.

It should be noted that in the training process of FIG. 3, the reconstruction 202 and the figure of merit determination 204 may in some implementations be performed as part of clinical tasks. For example, the training of FIG. 3 may employ historical PET imaging sessions stored in a Picture Archiving and Communication System (PACS). Each such PET imaging session typically includes reconstructing the images and extracting the figure(s) of merit from those images as part of the clinical assessment of the PET imaging session. Thus, this data may be effectively “pre-calculated” as part of routine clinical practice, and identified and retrieved from the PACS for use in training the deep learning transform 30.

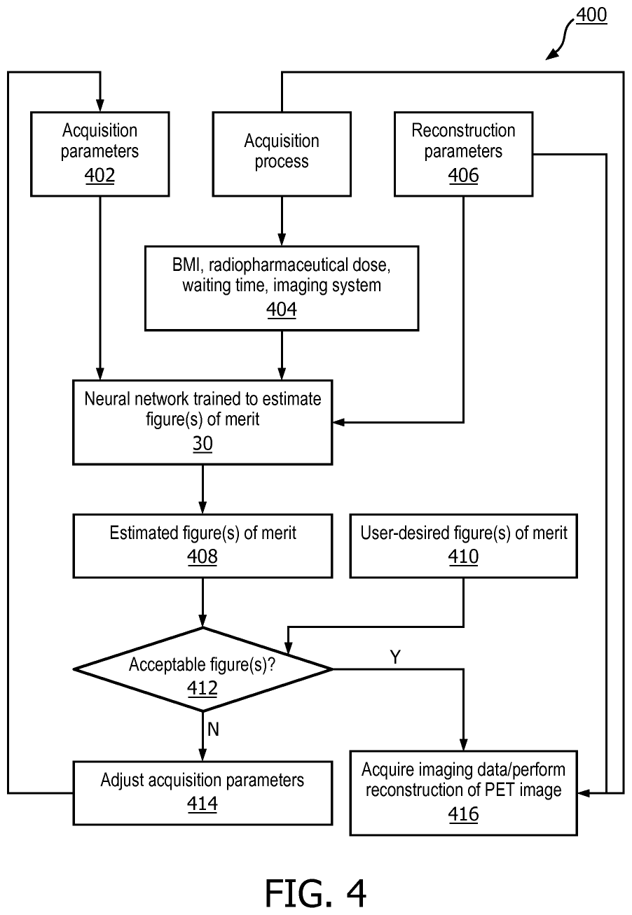

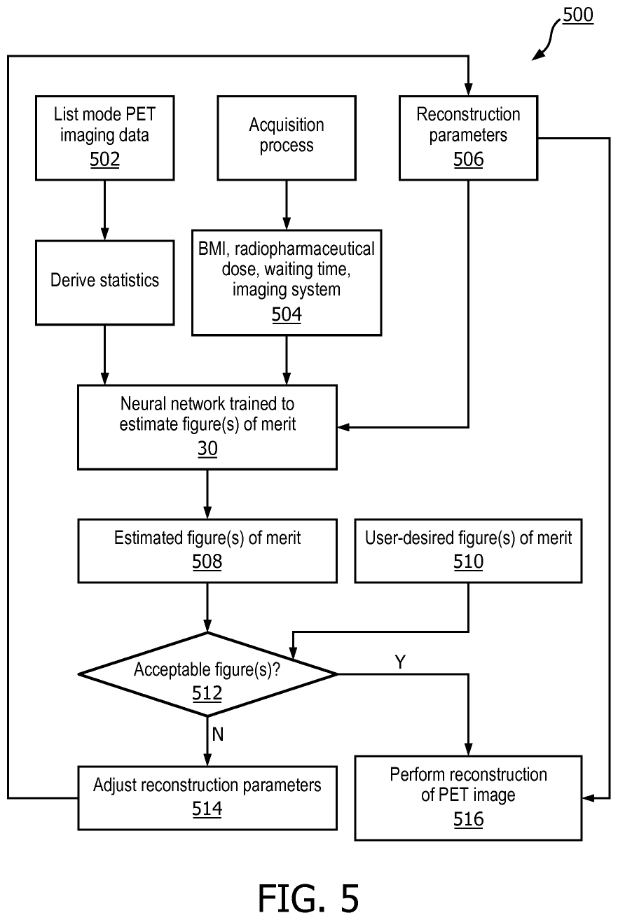

FIGS. 4 and 5 show more detailed flowcharts of the imaging method 100 is diagrammatically shown as a flowchart. FIG. 4 shows an embodiment of the imaging method 400 where the input data does not include imaging data. The inputs can include image acquisition parameter data (e.g., target portion to be imaged) 402, acquisition process data (e.g., dose and wait time, type of imaging system, imaging system specifications, etc.) 404, and reconstruction parameters 406. The inputs are input to the trained deep learning transform 30 (e.g. a neural network). At 408, the trained Neural Network 30 estimates one or more figures of merit (e.g., noise, SUV mean, and so forth) based on the inputs 402-406. At 410, user-desired figures of merit are input (e.g., via the one or more user inputs devices 22 of FIG. 1) to the trained Neural Network 30 of FIG. 4. At 412, the at least one electronic processor 20 is programmed to determine whether the estimated figures of merit are comparable (i.e., acceptable) relative to the user-desired figures of merit. If not, the acquisition parameters 402 are adjusted at 414 and the operations 402-412 are repeated. If the figures of merit are acceptable, then the at least one electronic processor 20 is programmed to, at 416, control the image acquisition device 12 to acquire imaging data and perform reconstruction of a PET image using the reconstruction parameters 406.

FIG. 5 shows another embodiment of the imaging method 500 where the input data to the neural network includes imaging data (but does not include any reconstructed image). At 502, statistics (e.g., total counts, counts/minute, and so forth) are derived from acquired list mode PET imaging data. The statistics are input to the Neural Network 30. Operations 504-512 of FIG. 5 substantially correspond to operations 404-412 of FIG. 4, and are not repeated here for brevity. At 514, if the figures of merit are not acceptable, then the reconstruction parameters are adjusted and used to re-acquire the list mode PET imaging data. If the figures of merit are acceptable, then the at least one electronic processor 20 is programmed to, at 516, perform reconstruction of a PET image using the reconstruction parameters 506.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory computer-readable medium storing instructions readable and executable by a workstation including at least one electronic processor to perform an imaging method, the method comprising:
estimating one or more figures of merit for a reconstructed image by applying a trained deep learning transform to input data including at least imaging parameters and not including a reconstructed image;
selecting values for the imaging parameters based on the estimated one or more figures of merit;
generating a reconstructed image using the selected values for the imaging parameters; and
displaying the reconstructed image.

2. The non-transitory computer-readable medium of claim 1, wherein
the input data includes imaging parameters comprising at least image reconstruction parameters and statistics of imaging data; and
the generating includes generating the reconstructed image by reconstructing the imaging data using the selected values for the image reconstruction parameters.

3. The non-transitory computer-readable medium of claim 2, wherein the input data does not include the imaging data.

4. The non-transitory computer-readable medium of claim 2, further comprising:
reconstructing training imaging data to generate corresponding training images;
determining values of the one or more figures of merit for the training images by processing of the training images;
estimating the one or more figures of merit for the training imaging data by applying the deep learning transform to input data including at least the image reconstruction parameters and statistics of the training imaging data; and
training the deep learning transform to match the estimates of the one or more figures of merit for the training imaging data with the determined values.

5. The non-transitory computer-readable medium of claim 1, wherein
the input data includes imaging parameters comprising at least image acquisition parameters; and
the generating includes acquiring imaging data using an image acquisition device with the selected values for the image acquisition parameters and reconstructing the acquired imaging data to generate the reconstructed image.

6. The non-transitory computer-readable medium of claim 5, wherein the input data does not include the acquired imaging data and does not include statistics of the acquired imaging data.

7. The non-transitory computer-readable medium of claim 5, further comprising:
reconstructing training imaging data to generate corresponding training images;
determining values of the one or more figures of merit for the training images by processing of the training images;
estimating the one or more figures of merit for the training imaging data by applying the deep learning transform to input data including at least the image acquisition parameters; and
training the deep learning transform to match the estimates of the one or more figures of merit for the training imaging data with the determined values.

8. The non-transitory computer-readable medium of claim 1 wherein the selecting comprises:
comparing the estimated one or more figures of merit with target values for the one or more figures of merit;
adjusting the imaging parameters based on the comparing; and
repeating the estimation of the one or more figures of merit for the reconstructed image by applying the trained deep learning transform (30) to input data including at least the adjusted imaging parameters and not including a reconstructed image.

9. The non-transitory computer-readable medium of claim 1, wherein the one or more figures of merit include a standardized uptake value (SUV) for an anatomical region.

10. The non-transitory computer-readable medium of claim 1 wherein the one or more figures of merit include a noise level for an anatomical region.

11. The non-transitory computer-readable medium of claim 10 wherein the trained deep learning transform is a trained support vector machine (SVM) or a trained neural network.

12. An imaging system, comprising:
a positron emission tomography (PET) image acquisition device configured to acquire PET imaging data; and
at least one electronic processor programmed to:
estimate one or more figures of merit for a reconstructed image by applying a trained deep learning transform to input data including at least image reconstruction parameters and statistics of imaging data and not including the reconstructed image;
select values for the image reconstruction parameters based on the estimated one or more figures of merit;
generate the reconstructed image by reconstructing the imaging data using the selected values for the image reconstruction parameters; and
control a display device to display the reconstructed image.

13. The imaging system of claim 12, wherein the input data does not include the imaging data.

14. The imaging system of claim 12, wherein the at least one electronic processor is programmed to:
reconstruct training imaging data to generate corresponding training images;
determine values of the one or more figures of merit for the training images by processing of the training images;
estimate the one or more figures of merit for the training imaging data by applying the deep learning transform to input data including at least the image reconstruction parameters and statistics of the training imaging data; and
train the deep learning transform to match the estimates of the one or more figures of merit for the training imaging data with the determined values.

15. The imaging system of claim 12, wherein the selecting comprises:
comparing the estimated one or more figures of merit with target values for the one or more figures of merit;
adjusting the imaging parameters based on the comparing; and
repeating the estimation of the one or more figures of merit for the reconstructed image by applying the trained deep learning transform to input data including at least the adjusted imaging parameters and not including a reconstructed image.

16. The imaging system of claim 12, wherein the one or more figures of merit include at least one of a standardized uptake value (SUV) for an anatomical region and a noise level for an anatomical region.

17. An imaging system, comprising:
a positron emission tomography (PET) image acquisition device configured to acquire PET imaging data; and
at least one electronic processor programmed to:
estimate one or more figures of merit for a reconstructed image by applying a trained deep learning transform to input data including at least image acquisition parameters and not including the reconstructed image;
select values for the image acquisition parameters based on the estimated one or more figures of merit;
generate the reconstructed image by acquiring imaging data using the image acquisition device with the selected values for the image acquisition parameters and reconstructing the acquired imaging data to generate the reconstructed image; and
control a display device to display the reconstructed image.

18. The imaging system of claim 17, wherein the input data does not include the acquired imaging data and does not include statistics of the acquired imaging data.

19. The imaging system of claim 17, wherein the at least one electronic processor is programmed to:
reconstruct training imaging data to generate corresponding training images;
determine values of the one or more figures of merit for the training images by processing of the training images;
estimate the one or more figures of merit for the training imaging data by applying the deep learning transform to input data including at least the image reconstruction parameters and statistics of the training imaging data; and
train the deep learning transform to match the estimates of the one or more figures of merit for the training imaging data with the determined values.

20. The imaging system of claim 17, wherein the selecting comprises:
comparing the estimated one or more figures of merit with target values for the one or more figures of merit;
adjusting the imaging parameters based on the comparing; and
repeating the estimation of the one or more figures of merit for the reconstructed image by applying the trained deep learning transform to input data including at least the adjusted imaging parameters and not including a reconstructed image.
